(19) United States
(12) Patent Application Publication
(10) Pub. No.: US 2019/0231631 A1
(43) Pub. Date: Aug. 1, 2019

(54) PRESSURE SENSING WALKING AID

(71) Applicant: CHRISTIAN D. CAMPILII, FISHKILL, NY (US)

(72) Inventor: CHRISTIAN D. CAMPILII, FISHKILL, NY (US)

(21) Appl. No.: 16/260,970

(22) Filed: Jan. 29, 2019

Related U.S. Application Data
(60) Provisional application No. 62/623,646, filed on Jan. 30, 2018.

(51) Int. Cl. A61H 3/02; A45B 3/08; A45B 3/04; A61H 3/04

(52) U.S. Cl. CPC A61H 3/02; A45B 3/08; A61B 2562/0247; A61H 3/04; A45B 3/04

(57) ABSTRACT

The present invention provides a pressure sensing walking aid that in some embodiments will provide a visual display for displaying the pressure data associated with a pressure sensor measuring a load supported by a walking device such as a walking aid, cane, crutch, walker or shoe.

### CROSS-REFERENCE TO RELATED APPLICATION

This application is a non-provisional application claiming the benefit of the prior filed U.S. provisional application No. 62/623,646 filed on Jan. 30, 2018 which is incorporated herein by reference.

### FIELD OF THE INVENTION

The present disclosure generally relates to waling aids that help improve the rehabilitation and mobility of those who are otherwise having difficulty walking safely based upon a physical injury, disability or medical condition. More particularly, the present invention pertains to a combination walking aid and sensor to determine the amount of weight supported by the walking aid.

### BACKGROUND OF THE INVENTION

Over six million people in the U.S. alone use some sort of mobility device, such as a cane, crutch or walker. Approximately four million of these use a cane as their mobility device. These walking aids, which help individuals (e.g., elderly persons, disabled persons) maintain balance or stability while walking, are generally known in the art. Conventional canes, walkers or walking aids are usually constructed of a lightweight tubular material and have a U-shaped frame which provides a place for an end user to grab to maintain balance or stability while walking. The typical walking aid is lightweight and intended to help balance while carrying only a portion of the user's weight and over time to decrease in the supported load, allowing the user to increase their carrying load over time. Increased dependence on the aid may lead to more permanent disability and more long-term reliance on the walking aid. Most users are not educated or taught on how to use their walking aid correctly and to only carry a portion of their weight on the aid. Because the cane, walker or walking aid requires use of one or both of hands on the handles, use on irregular or inclined surfaces also causes variation in the amount of load being carried by the aid.

Based in part on the foregoing challenges, there exists a need for a pressure-sensing walking aid which provides a visual indication of the load supported by the walking aid which will assist in the recovery and usage of the walking aid.

### SUMMARY OF THE INVENTION

The need for the present invention is met, to a great extent, by the present invention wherein in one aspect a pressure sensing walking aid is provided that in some embodiments will present a visual system for displaying the pressure associated with the supported load of an assistive walking device such as a walking aid, cane, crutch, walker or shoe. The pressure sensing walking aid generally provides stability during movement while displaying data associated with a measured quantity, the pressure sensing walking aid comprising a walking aid having a handle positioned near a visual display adapted for displaying data; a pressure sensing foot associated with at least one sensor and in communication with said visual display for transmitting data associated with a measured quantity to said display; an elongated structure associated with said walking aid and extending between said handle and said pressure sensing foot; and said pressure sensing foot configured for receipt of said elongated structure.

The pressure sensing walking aid includes a walking support structure and a display in communication with a pressure sensor. Generally, the pressure sensor includes a pair of plates associated with a sensor, located near the ground and integrated into the elongated support structure. Generally, the sensor produces a signal which varies based upon the measured load placed upon the walking support structure. Generally, the display includes a display screen with a display window for displaying data from the sensor and in one embodiment displays pressure data from a pressure sensor.

Certain embodiments of the invention are outlined above in order that the detailed description thereof may be better understood, and in order that the present contributes to the art may be better appreciated. There are, of course, additional embodiments of the invention that will be described below and which will form the subject matter of any claims appended hereto.

In this respect, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein as well as the abstract are for the purposes of description and should not be regarded as limiting.

As such, those skilled in the relevant art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention. Though some features of the invention may be claimed in dependency, each feature has merit when used independently.

Various objects and advantages of the present invention will become apparent from the following description taken in conjunction with the accompanying drawings wherein are set forth, by way of illustration and example, certain embodiments of this invention. The drawings submitted herewith constitute a part of this specification, include exemplary embodiments of the present invention, and illustrate various objects and features thereof.

### BRIEF DESCRIPTION OF THE DRAWINGS

Further features of the present invention will become apparent to those skilled in the art to which the present invention relates from reading the following description with reference to the accompanying drawings, in which a better understanding of the present invention is depicted, in which:

FIG. 1 is a cross sectional elevation of an exemplary embodiment of the integrated pressure sensor associated a walking support structure.

FIG. 2 is a fragmented side elevation of an exemplary integrated pressure sensing foot.

FIG. 3 is an exploded fragmented side elevation of an exemplary integrated pressure sensing foot according to FIG. 2.

FIG. 4 is an exemplary cane embodiment with the exemplary integrated pressure sensing foot of FIG. 2.

FIG. 5 is an exemplary crutch embodiment with the exemplary integrated pressure sensing foot of FIG. 2.

FIG. 6 is an exemplary walker embodiment with the exemplary integrated pressure sensing foot of FIG. 2.

FIG. 7 is an exemplary shoe embodiment with an alternative embodiment of the integrated pressure sensor.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

Accordingly, the above problems and difficulties are obviated, at least in part, by the present system which provides a pressure sensing walking aid 10 shaped as a walking aid 11 with an elongated structure 12 and configured for movement. A stabilizer 30 is mounted to the elongated structure 12. The elongated structure 12 extends between a pressure sensing foot 48 and a handle 4, an electronic visual display 18 mounted along the elongated structure 12 and in electronic communication with the pressure sensing foot 48. Generally, the pressure sensing walking aid 10 provides additional stability during movement while allowing measuring, monitoring, displaying and providing feedback regarding a supported load. The pressure sensing walking aid 10 is generally adapted for use in a variety of walking aid embodiments with variously configured support structures 12 also referred to herein as an elongated support structure.

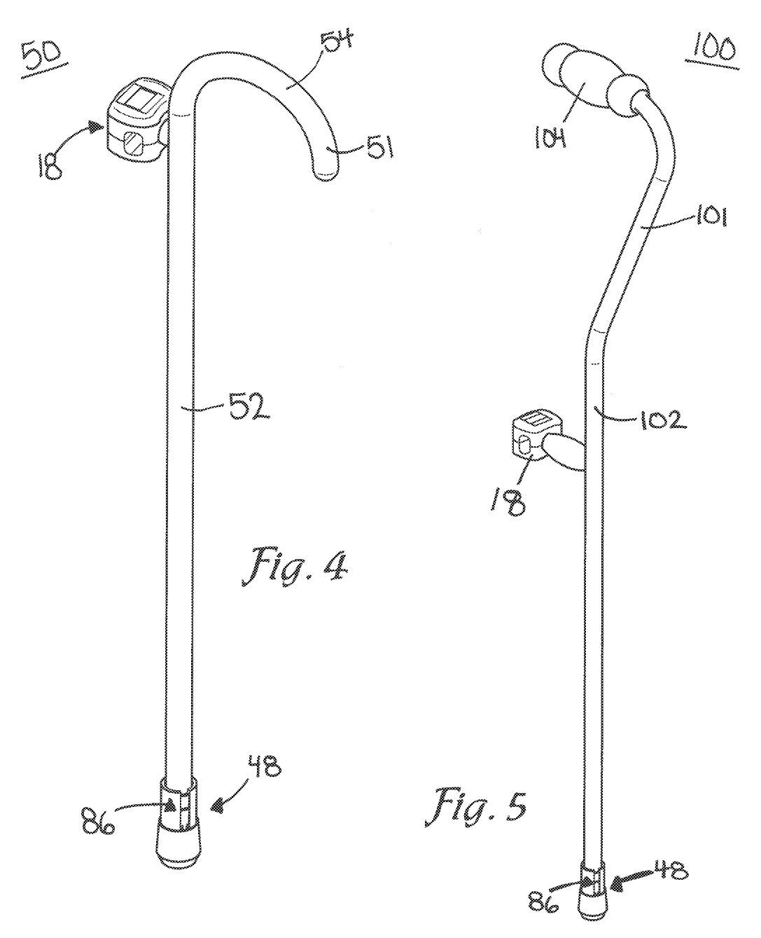

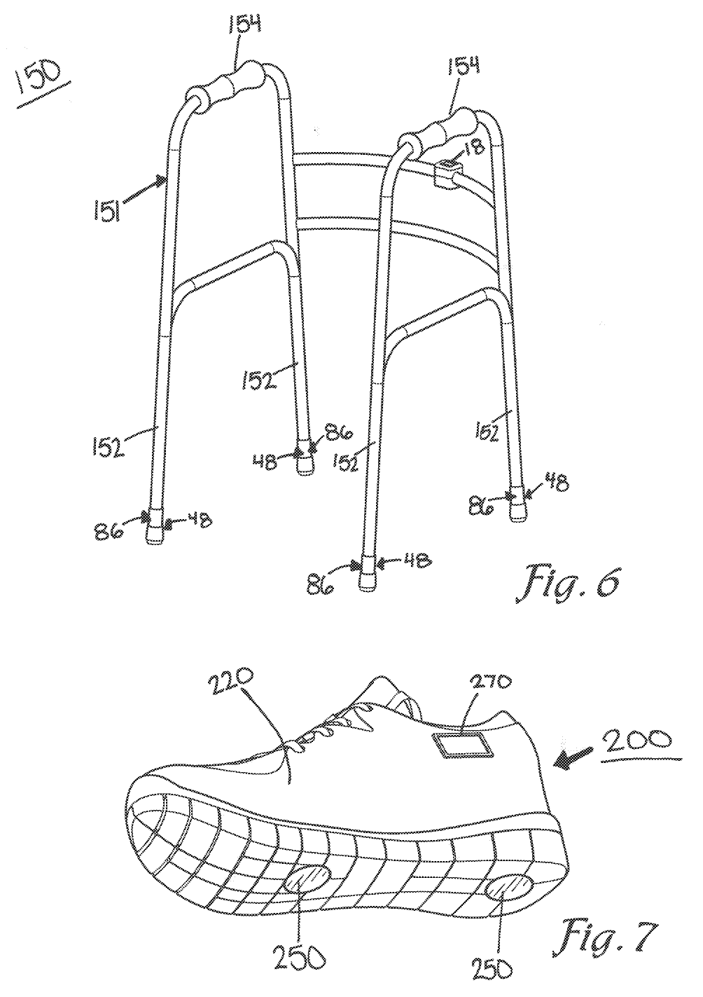

The pressure sensing walking aid 10 is depicted in FIGS. 4-6 with different embodiments of the walking aid, such as, an embodiment of a cane 50 depicted in FIG. 4, an embodiment of a crutch 100 depicted in FIG. 5, an embodiment of a walker 150 depicted in FIG. 6 and an embodiment of a shoe 200 depicted in FIG. 7.

Generally, the pressure sensing walking aid 10 includes the structure of a walking aid 11 having the handle 4 and at least one elongated support structure 12. The pressure sensing foot 48 is configured for receipt of one end of the elongated structure 12. The pressure sensing foot 48 including a pressure sensor 20 at least partially received by the stabilizer 30, the pressure sensor 20 being in electrical communication with a display 18 which is mechanically attached to the walking aid 11 along a portion of the elongated support structure 12. The handle 4 is generally positioned along the elongated support structure 12 and is in communication with the pressure sensing foot 48 for selective movement of the walking aid 11.

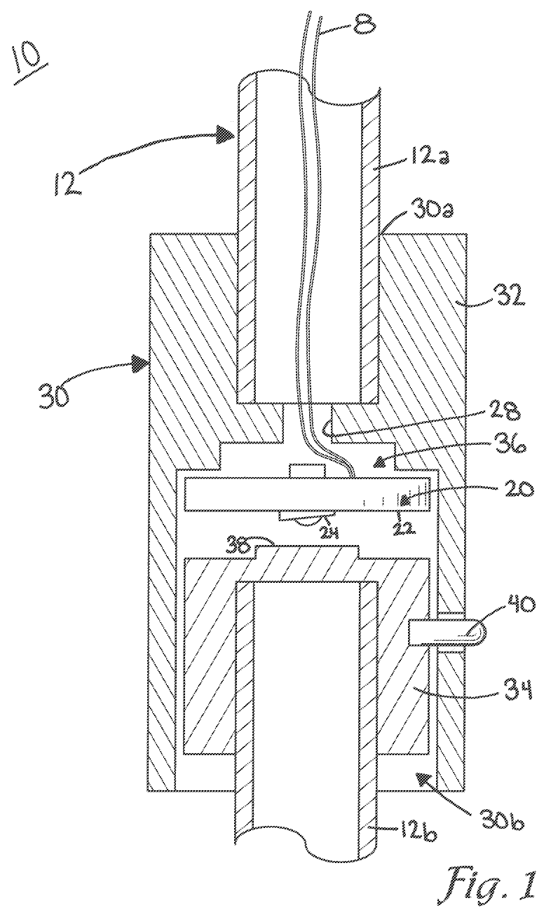

As illustrated in FIG. 1, stabilizer 30 is mechanically secured to the elongated support structure 12 and includes an internal structure 32 that at least partially encloses the pressure sensor 20, a portion of the elongated support structure 12 being received at opposite ends of the stabilizer 30. Generally, the stabilizer upper end 30a is vertically superior and opposite stabilizer lower end 30b. The elongated support structure 12 has an upper end 12a associated with the stabilizer upper end 30a which is spaced from a lower end 12b at stabilizer lower end 30b. The stabilizer lower end 30b is generally adapted for receipt by the pressure sensing foot 48.

As further described below, one embodiment of the pressure sensing walking aid is depicted as a cane walking aid 51 in FIG. 4 with an exemplary cane 50 being illustrated and providing the handle 54 and support structure 52. Other cane configurations which are generally known may be utilized as part of the embodiment of the cane walking aid 51.

As further described below, a second embodiment of the pressure sensing walking aid is depicted as a crutch walking aid 101 in FIG. 5 with an exemplary crutch 100 being illustrated and providing a handle 104 and support structure 102. Other configurations of generally known crutches 100 may be utilized as part of the embodiment of the crutch walking aid 101.

As further described below a third embodiment of the pressure sensing walking aid is depicted as a walker walking aid 151 in FIG. 6 with an exemplary walker 150 being illustrated and providing a handle and support structure 152. Other configurations of generally known walkers 150 may be utilized as part of the embodiment of the walker walking aid 151.

As further described below, a fourth embodiment of the pressure sensing walking aid is depicted as a shoe walking aid 201 in FIG. 7 with an exemplary shoe 200 being illustrated. Other generally known shoe 200 configurations may be utilized as part of the embodiment of the shoe walking aid 201.

Generally, the configuration of the walker aid 11 for use in the current invention comprises a handle 4 and support structure 12 with at least one supporting member which terminates at the pressure sensing foot 48. The walker aid 11 may include a number of known walking aid configurations such as the cane, walker (wheeled or unwheeled), crutch or the like providing sufficient stability and support for engagement by the user during locomotion along a ground engaging surface (not shown) as the user moves from one location to another. The handle 4 is located along the support structure 12, while the pressure sensing foot 48 receives a terminal end of the support structure 12 and presents a ground engaging surface for movement along the ground.

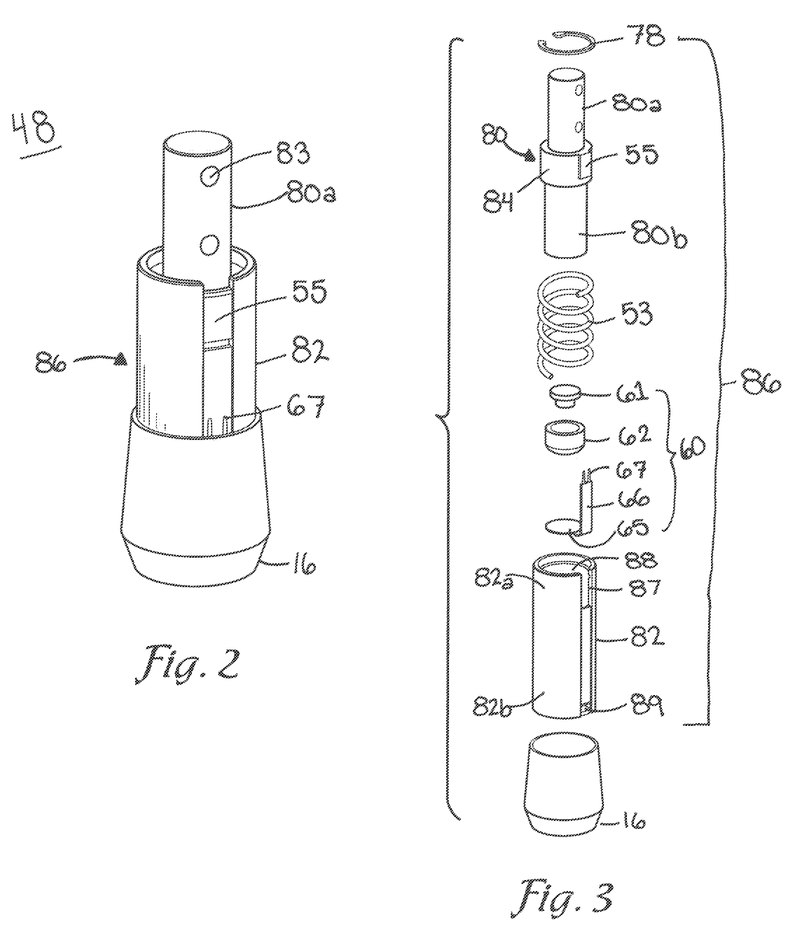

Depending on the desired configuration of the walking aid 11, at least one ground engaging surface 16 may be provided. Generally, the ground engaging surface 16 is adapted for receiving lower end 12b, the ground engaging surface 16 depicted in FIG. 2 having a cylindrical body extending from a circular cap which together encircle the lower end 12b during engagement. The ground engaging surface 16 is fabricated with a durable, abrasion and weather-resistant surface adapted for prolonged use indoors and outdoors. Depending on the desired use, the ground engaging surface may also have less or more frictional resistance to provide more or less traction during movement. The ground engaging surface 16 may also be removed and replaced as necessary for maintenance. An exemplary embodiment of the ground engaging surface 16 is illustrated as having a cavity for receiving a portion of the alternative stabilizer 86 and for frictional engagement with an underlying surface to prevent unwanted movement of the walking aid 11. The ground engaging surface 16 may be fabricated from a frictional material such as, but not limited to, rubber or plastic and generally is between 1″ to 2″ in diameter with an inner diameter having sufficient dimensions for receipt of the lower support structure 12b.

FIG. 1 depicts an embodiment of the stabilizer 30. Generally, the stabilizer 30 provides additional stability to the walking aid during locomotion and reducing the reactionary force caused from the impact of the walking aid 11 upon the ground (not shown) to the handle 4 along the elongated support structure 12, reducing vibrational and environmental forces exerted upon the user of the walking aid 11.

In the depicted embodiment of FIG. 1, the internal structure 32 associated with the stabilizer 30 receives the inner carrier 34 while providing a compressible gap 36 between the upper end 12a and the lower end 12b for receiving and retaining the pressure sensor 20.

As the walking aid 11 is moved along the ground (not shown), the reactionary force causes the inner carrier 34 to operate reciprocally, like a piston, during movement of the walking aid 11 upon the ground (not shown). Reciprocal operation of the inner carrier 34 directs the inner carrier 34 towards the pressure sensor, compressing the compressible gap 36. The inner carrier 34 includes a radial projection 38 which is on the inner carrier 34, opposite the lower end 12b, near the pressure sensor 20. As the radial projection 38 reacts to the reactionary force exerted upon the lower end 12b, the pressure sensor 20 exhibits a change.

In one embodiment, the pressure sensor 20 may be utilize an accelerometer, a hydrostatic pressure sensor or a piezoelectric sensor which deform as the pressure changes. The deformation may be converted to an electrical signal which is then transmitted via the electrical wires 8 to the display 18. A power source may be supplied to the sensor from the display 18 or it may be associated with the sensor 20. A microprocessor or logic circuit (not shown) may be utilized as part of the display 18 to provide the relevant information to allow the user to measure, monitor, display and provide relevant feedback regarding the supported load upon the walking aid 11 during movement.

The embodiment depicted in FIG. 1 includes internal structure 32 with a stabilizer lower receiver 30b and a stabilizer upper end 30a. The stabilizer lower receiver 30b has sufficient internal diameter for receipt of the inner carrier 34. The stabilizer upper end 30a has an internal diameter for receipt of the upper end 12a. Greater stability is provided by centering the inner carrier 34 within the stabilizer lower receiver 30b and redistributing the supported load and reactionary forces over a centralized larger surface. The inner carrier 34 is floated within the stabilizer lower receiver 30b near a centralized gap 36 which isolates at least a portion of the elongated support structure 12 while also allowing for connection to the internal structure 32, centrally aligning the upper end 30a with the lower receiver 30b. By providing an inner carrier 34 with a larger mass and centering it within the stabilizer lower receiver 30b, the center of mass of the walking aid 11 is redistributed so that the magnitude of the reactionary force exerted upon the user is reduced without compromising the deflection experienced by the pressure sensor 20 during movement.

The stabilizer 30 of FIG. 1 generally extends between two ends of the support structure 12, an upper end 12a and a lower end 12b and encircling the elongated support 12. The internal structure 32 generally presents a radially extending central receiving chamber which is adapted for reciprocal receipt of the inner carrier 34. Generally, the inner carrier 34 is associated with the elongated support structure 12. As depicted in the illustrated embodiment of FIG. 1, the inner carrier 34 generally encircles the lower end 12b, but it could alternatively encircle the upper end 12a if the pressure sensor 20 is flipped.

The stabilizer upper end 30a may frictionally retain the upper end 12a or it may be adapted for mechanical fastening using fasteners or complementary grooved structures like a threaded fastener, if desired, to retain the upper end 12a within the stabilizer upper end 30a. As depicted, electrical wires 8 from the pressure sensor 20 are transmitted through the upper end 12a, facilitating electrical communication between the display 18 and the sensor 20.

The central receiving chamber 36 has sufficient dimensions and structure for housing the sensor 20, such as a load cell sensor, sufficient spaced from the inner carrier 34 or piston for accurately measuring the weight of the supported load. Generally, the pressure sensor 20 includes a flexible circular plate 22 with a centrally located deflection sensor 24 which measures the load-induced deflection of the flexible circular plate 22. The radial projection 38 is spaced opposite the lower end 12b and, during operation, is reciprocated towards the upper end 12a. Located between the inner carrier 34 and the internal structure 32, the pressure sensor 20 depicted in FIG. 1 includes a deflection sensor 24 in electrical communication with display 18. The deflection sensor 24 or other sensing device can determine the amount of force exerted upon the pressure sensor 20 during use of the pressure sensing walking aid 10 by measuring the deflection exhibited by the pressure sensor as the inner carrier 34 extends rearwardly.

In the embodiment of the pressure sensor 20 depicted in FIG. 1, the pressure sensor 20 is a load cell type sensor which senses the parallel force exerted upon the elongated support structure 12. In one exemplary process for sensing the load force, a pressure may be applied to the elongated support structure 12, which is extended along the upper end 12a through the internal structure 32 along the lower end 12b to the pressure sensing foot 48. Through engagement between the ground (not shown) and the pressure sensing foot 48, a reactionary force is exerted upon the lower end 12b to the inner carrier 34, which drives the inner carrier 34 rearwardly within the central receiving chamber 36. As the inner carrier 34 extends rearwardly within the receiving chamber 36, the radial projection 38 is directed rearwardly towards the deflection sensor 24 overlying the flexible circular plate 22. A deflection caused by the radial projection 38 to the flexible circular plate 22, generates a signal whose magnitude of deflection corresponds to the reactionary force exerted upon the lower end 12b. The signal corresponding to the pressure is then transmitted along the electrical wires 8 to the display 18 or can be sent wirelessly via known wireless technologies.

A fastener 40, also referred to as a fastening assembly, is depicted in FIG. 1 in engagement between the inner carrier 34 and the outer stabilizer receiver 32. Generally, the fastening assembly 40 provides limited engagement of the inner carrier 34 to the outer stabilizer receiver 32 and at least partially limits independent reciprocal movement of the inner carrier 34 within the outer stabilizer receiver 32. As depicted in FIG. 1, one embodiment of the fastening assembly 40 utilizes a slotted opening 42 in the inner carrier 34 adapted for receipt of a locking pin 44 extendable through a passage 46 in the outer stabilizer receiver 32. In addition, the passage 46 allows for movement of the retained locking pin 44 during operation of the combination pressure sensing walking system 10.

As further depicted in FIG. 1, the embodiment of the central receiving chamber 36 includes a pair of rearward supports 26. Generally, the reward supports 26 provide sufficient support for the sensor 20 while permitting desired deformation of the flexible circular plate 22. In addition, the reward supports 26 present access to an internal passageway 28, for running the electrical wires 8 to the display 18 for example, which extend between the central receiving chamber 36 associated with the outer stabilizer receiver 32 and the support structure 12 at the upper end 12a.

FIGS. 2-3 illustrates an embodiment of the pressure sensing foot 48 which includes a vertically adjustable alternative stabilizer embodiment 86 with a substantially cylindrical body, encircled by an alternative inner carrier 84 depicted as a circular ring 52 extending radially from the outer surface of the alternative stabilizer 86. As further depicted in FIG. 3, the inner carrier 84 also includes a slot 55 projecting radially from the inner carrier 84 and depicted as being rectangular.

The depicted alternative stabilizer 86 includes a cylindrical structure 80 with a cylindrical upper end 80a opposite a cylindrical lower end 80b, medially joined by the alternative inner carrier 84. Cylindrical receiver 82 presents an internal lumen or passageway 88 which is adapted for receiving the alternative inner carrier 84 which separates the cylindrical structure 80 from the cylindrical receiver 82 along with the sensor assembly 60. In the embodiment depicted in FIG. 3, an upper portion 82a includes an elongated groove 87 and a lower portion 82b includes a slotted opening 89. The elongated groove 87 is adapted for receipt of the inner carrier 84 while the slotted opening 89 at least partially receives the sensor assembly 60.

The cylindrical receiver 82 presents the elongated groove 87 extending downward from engaged receipt of slot 55. The combination groove 87 and slot 55, present an alternative fastening assembly allowing engagement between the alternative inner carrier 84 to the cylindrical receiver 82. Alternatively, or in combination, a lock-ring 78 may be utilized to also secure the cylindrical structure 80 to the cylindrical receiver 82. The alternative inner carrier 84 generally includes a sensor assembly 60 adapted for receipt by the alternative internal passageway 88 has sufficient dimensions and structure for housing the alternative inner carrier 84 along with alternative sensor 21.

A plurality of adjusters 83 are depicted in FIGS. 2-3 spaced along the alternative stabilizer 86 along the cylindrical upper end 80a, which allows for vertical adjustment of the alternative stabilizer 86 received by the pressure sensing foot 48. The adjusters 83 provide adjustable structure for vertical configuration of the elongated structure 12 as desired. The adjusters 83 may utilize a number of fixed or releasable complementary mechanical fasteners associated with the elongated support structure 12 which may allow for height adjustment of the pressure sensing foot 48. The adjustable cylindrical structure 80 allows the pressure sensing foot 48 to be easily retrofit or installed on the end of a variety of existing and new walking aids.

A biasing member 53, such as a spring, is positioned between alternative inner carrier 84 and cylindrical receiver 82 and generally assists in transferring at least a portion of the downwardly directed received force resulting from the support structure 12 to the sensor assembly 60 associated with the pressure sensing foot 48. During locomotion, the handle 4 receives the downwardly directed force associated with the walking aid 11 which is generally distributed along the elongated support structure 12. The alternative inner carrier 84 extends radially from the cylindrical structure 80 at the junction of the cylindrical upper end 80a and the cylindrical lower end 80b.

The alternative inner carrier 84 receives and transmits a portion of the received force to the biasing member 53 which is then transmitted to the sensor assembly 60. In addition to transferring at least a portion of the received force to the sensor assembly 60, the biasing member 53 at least partially contracts in response to the received force and responds with an opposite, upwardly directed reactionary force applied against the alternative inner carrier 84.

The magnitude of the oppositely directed reactionary force varies based on the differential distance and the physical characteristics of the biasing member 53 in accordance with Hooke's Law in which F=kX, F corresponding to the force needed to compress or extend the bias member, k being a constant dependent upon the physical characteristics of the biasing member 53 and X representing the linear distance the biasing member 53 is compressed or extended.

The alternative inner carrier 84 encircles the cylindrical structure 80 and includes a radial projection for engagement of the biasing member 53. The alternative inner carrier 84 depicted in FIGS. 2-3 includes an outwardly extending slot 55 which is adapted for centrally aligning the cylindrical structure 80 within the cylindrical receiver 82 for alignment during reciprocal operation of the alternative stabilizer 86.

Generally, the cylindrical structure 80, alternative inner carrier 84 associated with the alternative stabilizer 86 are rigid and capable of handling a supporting load force associated with a user ranging from under 100 to over 500 pounds and may be fabricated from a metal, plastic, organic or inorganic materials which are able to support the received force.

The alternative inner carrier 84 is adapted for receipt of the sensor assembly 60 which may include a first magnetic structure 61 coupled to a second magnetic structure 62 associated with the alternative pressure sensor 21 which are adapted for telescopic receipt by the internal passageway 88. The sensor assembly 60 generally measures the force that is placed on the support structure 12 and may include vertical movement. The alternative pressure sensor 21 may include a load cell sensor to measure the deflection experienced by the sensor assembly 60 as the pressure sensor walking aid 10 is utilized for supporting a load during movement along the ground.

In operation, the sensor assembly 60 is received within the internal passageway 88 for engagement with the magnet structure 62. The sensor assembly 60 is configured for receipt by the internal passageway and as depicted is circular. The sensor assembly 60 is configured to allow the sensor projection 66 to extend outwardly from the slotted opening 89 for reciprocal receipt by the elongated groove 87. Generally, the sensor receiver 65 is disk shaped in accordance with the cylindrical internal passageway 88 and the sensor projections are configured to receive and transmits sensor data via the integrated wires 67 to the display 18. The sensor assembly 60 is generally weather resistant for use in an outdoor and indoor environment and will be capable of achieving the necessary certifications for the desired use.

Channel 87 extends longitudinally along the cylindrical sidewall of the alternative stabilizer 86 with the upper portion 82a adapted for vertical receipt of the slot 55 and at the lower portion 82b includes a slotted opening 89. The channel 87 includes a lower limit associated with the upper portion 82a which limits the vertical movement of the received slot 55. Controlling the vertical movement of the received slot 55, allows for controlled placement of the alternative inner carrier 84 in relation to the alternative stabilizer 86. The slotted opening 89 allows for at least partial receipt of the alternative sensor 21.

As further depicted in FIG. 3, the alternative sensor 21 includes a sensor receiver 65 normal to a sensor projection 66 which includes a pair of integrated wires 67 extending from the sensor receiver 65 to the sensor projection 66. In the depicted embodiment, the sensor projection 66 extends outward and upward from the sensor receiver 65 allowing for vertical movement of the alternative sensor 21 with the sensor projection 66 traveling within the channel 87 as the walking aid 11 makes reciprocal movement during locomotion. Generally, the alternative sensor 21 may be connected to the display 18 via wired connection with the integrated wires 67 or wirelessly via known wireless protocols like Bluetooth.

FIG. 4 illustrates an embodiment of the pressure sensing walking aid 10 utilizing the cane walking aid 51 illustrated with the alternative stabilizer 86 in electrical communication with display 18 secured to the elongated support structure 52 near handle 54. The elongated support structure 52 associated with the cane walking aid 51 generally extends from the handle 54 to the pressure sensing foot 48.

FIG. 5 illustrates an alternative embodiment of the pressure sensing walking aid 10 utilizing the crutch walking aid 101 illustrated with the alternative stabilizer 86 in electrical communication with display 18 secured to the elongated support structure 12 near handle 104. The elongated support structure 102 associated with the crutch walking aid 101 generally extends from the handle 104 to the pressure sensing foot 48.

FIG. 6 illustrates an alternative embodiment of the walker walking aid 151, having an alternative handle 154 and an alternative elongated support structure 152 adapted for receipt by the pressure sensing foot 48 associated with each terminal end of the elongated support structure 152. Each pressure sensing foot 48 is in electrical communication with the display 18 which may provide information about an individual pressure sensing foot 48, or a plurality of pressure sensing feet 48 for use during locomotion. Each pressure sensing foot 48 may optionally include the ground engaging surface 16.

FIG. 7 illustrates another alternative embodiment of a shoe walking aid 200, adapted for use with a second alternative pressure sensing foot 250, in which the aforementioned features are housed within an internal cavity (not shown) associated with the shoe 200 and the alternative display 270 is located within a side panel 220 associated with the shoe 200. The alternative display 270 is generally in electrical communication with the internally housed pair of second alternative pressure sensing foot 250.

It is to be understood that while certain forms of the present invention have been illustrated and described herein, it is not to be limited to the specific forms or arrangement of parts describer herein. Other arrangements or embodiments, changes and modifications not precisely set forth, which can be practiced under the teachings of the present invention are to be understood as being included within the scope of this invention as set forth in the claims below.

What is claimed and desired to be secured by Letters Patent:

1. A pressure sensing walking aid providing stability during movement while displaying data associated with a measured quantity, the pressure sensing walking aid comprising:
a walking aid having a handle positioned near a visual display adapted for displaying data;
a pressure sensing foot associated with at least one sensor and in communication with said visual display for transmitting data associated with a measured quantity to said display;
an elongated structure associated with said walking aid and extending between said handle and said pressure sensing foot; and
said pressure sensing foot configured for receipt of said elongated structure.

2. The pressure sensing walking aid of claim 1 further comprising a stabilizer mounted to said elongated structure and at in at least partial receipt of said sensor.

3. The pressure sensing walking aid of claim 2 wherein said elongated structure is received by opposite sides of said stabilizer.

4. The pressure sensing walking aid of claim 2 wherein said stabilizer is received by said pressure sensing foot.

5. The pressure sensing walking aid of claim 1 wherein said walking aid is selected from the group consisting of a cane, crutch, and walker.

6. The pressure sensing walking aid of claim 1 further comprising a ground engaging surface.

7. The pressure sensing walking aid of claim 6 wherein said elongated structure further comprises an upper end and a lower end, said upper end at least partially extending between said handle and said pressure sensing foot and said lower end at least partially extending between said lower end and said ground engaging surface.

8. The pressure sensing walking aid of claim 1 wherein said sensor is a pressure sensor configured for measuring and transmitting pressure data to said visual display.
